(19) United States
(12) Patent Application Publication
(10) Pub. No.: US 2019/0203181 A1
(43) Pub. Date: Jul. 4, 2019

(54) METHOD FOR PREPARING MESENCHYMAL STEM CELL PREPARATION BY USING ARTHROSCOPIC IRRIGATION SOLUTION

(71) Applicant: Lugene Technology Co., Ltd., Shenzhen (CN)

(72) Inventors: Jun LI, Shenzhen (CN); Guangqian ZHOU, Shenzhen (CN)

(21) Appl. No.: 16/325,750

(22) PCT Filed: Aug. 17, 2016
(86) PCT No.: PCT/CN2016/000458

§ 371 Date: Feb. 15, 2019

(51) Int. Cl. C12N 5/0775; A61K 35/28; A61L 27/38

(52) U.S. Cl. CPC C12N 5/0662; A61L 27/3834; A61K 35/28

(57) ABSTRACT

A method for preparing a mesenchymal stem cell preparation by using an arthroscopic irrigation solution, comprising: 1) collecting an irrigation solution in an arthroscopic surgery; 2) extracting and expanding stem cells; 3) identifying characteristics of the stem cells; 4) preparing a stem cell preparation; and 5) controlling the quality of the stem cell preparation.

### TECHNICAL FIELD

The present disclosure relates to mesenchymal stem cells, and particularly to a method for preparing a mesenchymal stem cell preparation by using an arthroscopic flushing solution.

### BACKGROUND

With the development of cell regenerative medicine, stem cell therapy has made a revolutionary breakthrough and is one of the most concerned and fastest-developing fields in modern medicine, providing new hopes for diseases that cannot be cured by various conventional methods. However, there is a lack of seed cells with abundant sources, convenient collection, and stable activity in both cell transplantation and tissue engineering. The selection of suitable seed cells is a top priority for stem cell therapy. An arthroscopic surgery flushing solution contains a large number of mesenchymal stem cells (MSCs) from cartilage, synovial fluid, synovium, and joint capsule washed out from the joint cavity. By collecting an flushing solution in an arthroscopic surgery from which MSCs are extracted, cultured, and expanded, and establishing a cell bank, it is considered to be the most convenient and effective method to obtain MSCs. In addition, the obtained MSCs can be efficiently utilized as seed cells for cell therapy or cartilage tissue engineering. After MSCs are prepared into clinical-grade applicable stem cell preparations, they have broad market application prospects.

### SUMMARY

In view of the shortcomings of the existing techniques and the needs of the market, it is an object of the present disclosure to provide a method for preparing a mesenchymal stem cell preparation using an arthroscopic flushing solution. In order to provide a solution for the problem in articular cartilage damage and degenerative diseases in tissue engineering and cell transplantation, i.e. the lack of seed cells with convenient collection, abundant sources, stable activity and high specificity, MSCs are isolated and extracted from a conventionally discarded arthroscopic flushing solution and expanded to provide reliable seed cells for the regenerative repair and treatment of articular cartilage damage and degeneration.

A technical solution adopted by the present disclosure to solve the technical problem thereof is to provide a method for preparing a mesenchymal stem cell preparation by using an arthroscopic flushing solution including:

1) collecting a flushing solution in an arthroscopic surgery: collecting the flushing solution by a sterile collection bottle during the arthroscopic surgery and storing the flushing solution for use;
2) extracting and expanding stem cells: dispensing the collected arthroscopic flushing solution on a clean bench and centrifuging the arthroscopic flushing solution, discarding a supernatant, adding DMEM medium containing 2% by weight of serum replacement to a resulting precipitate and re-suspending the cells, inoculating the cells into a culture flask, replacing the medium for the first time after 45 h to 50 h, and then replacing the medium every 3 days for 12 days to 16 days to obtain adherent cells, passaging the cells when cell clones are in contact with each other; purifying and expanding the cells after passages, using the 6th passage cells for subsequent steps;
3) identifying characteristics of the stem cells: detecting a proliferative ability of the stem cells by using CCK-8 reagent; using induction solutions for adipogenic, osteogenic, and chondrogenic induction of the stem cells, identifying a multi-directional differentiation ability of the stem cells by staining; identifying mesenchymal stem cell-specific antigens by analyzing stem cell surface markers CD34⁻, CD45⁻, CD73⁺, CD90⁺, and CD105⁺ with the cells being negative when expressions of CD34⁻ and CD45⁻ are less than 1% and the cells being positive when expressions of CD73⁺, CD90⁺, and CD105⁺ are greater than 95%;
4) preparing a stem cell preparation: dispensing the resulting stem cells in a serum-free medium and storage additives thereof at a concentration of 1×10⁶ cells/ml to 1×10⁸ cells/ml and storing the stem cell preparation in a cell bank;
5) controlling quality of the stem cell preparation: performing tests selected from the group consisting of stem cell survival rate and growth activity, purity and homogeneity, sterility test and mycoplasma detection, detection of intracellular exogenous pathogenic factors, endotoxin detection, and detection of residual amounts of culture medium and other additive components according to “Guidelines for Quality Control and Preclinical Research of Stem Cell Preparation (Trial)” issued by China on Aug. 21, 2015 with all qualified preparations as finished products.

The present disclosure has following advantageous effects: compared with MSCs from other sources, MSCs isolated and extracted from the conventionally discarded arthroscopic flushing solution and expanded have the advantages of collection convenience, abundant sources, stable activity, and high specificity, which will become a new type of high-quality seed cells for cartilage regenerative repair and can provide a large number of stem cell resources for clinical and scientific research. The preparation of clinical-grade applicable stem cell preparations and the establishment of stem cell banks can provide seed cell resources for stem cell therapy, and the industrialization and marketization prospects are very broad.

### BRIEF DESCRIPTION OF THE DRAWINGS

FIG. 1 shows morphology of stem cells in an isolated culture of arthroscopic flushing solution-derived stem cells of the present disclosure under a light microscope on day 1.

FIG. 2 shows morphology of stem cells in the isolated culture of arthroscopic flushing solution-derived stem cells of the present disclosure under a light microscope on day 3.

FIG. 3 shows morphology of stem cells in the isolated culture of arthroscopic flushing solution-derived stem cells of the present disclosure under a light microscope on day 7.

FIG. 4 shows the staining after formation of stem cell colonies.

FIG. 5 shows the proliferative ability of stem cells detected by CCK-8.

FIG. 6 shows the adipogenic differentiation induction of arthroscopic flushing solution-derived stem cells.

FIG. 7 shows the osteogenic differentiation induction of arthroscopic flushing solution-derived stem cells.

FIG. 8 shows the chondrogenic differentiation induction of arthroscopic flushing solution-derived stem cells.

FIG. 9 is an identification diagram of surface markers of arthroscopic flushing solution-derived stem cells.

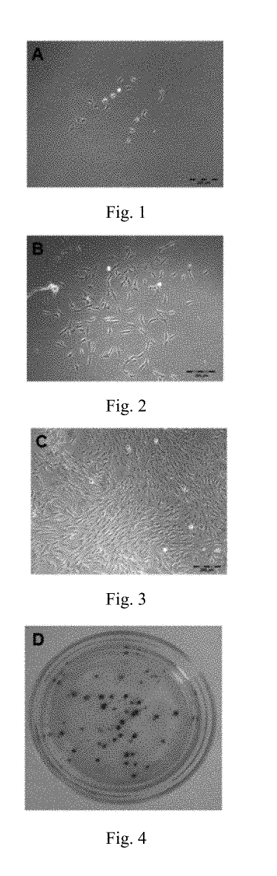

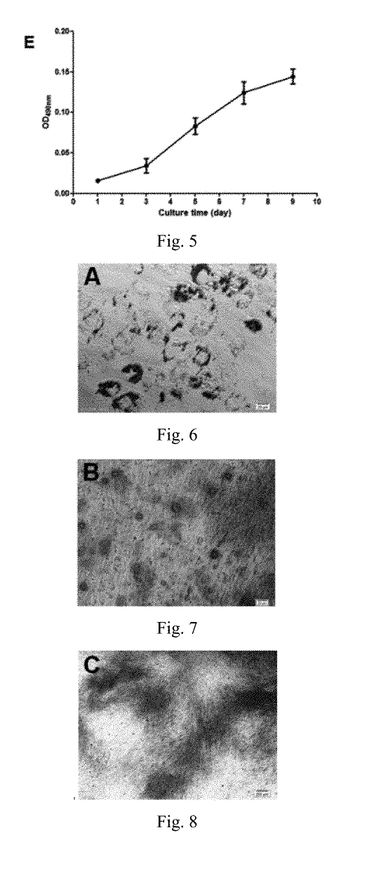

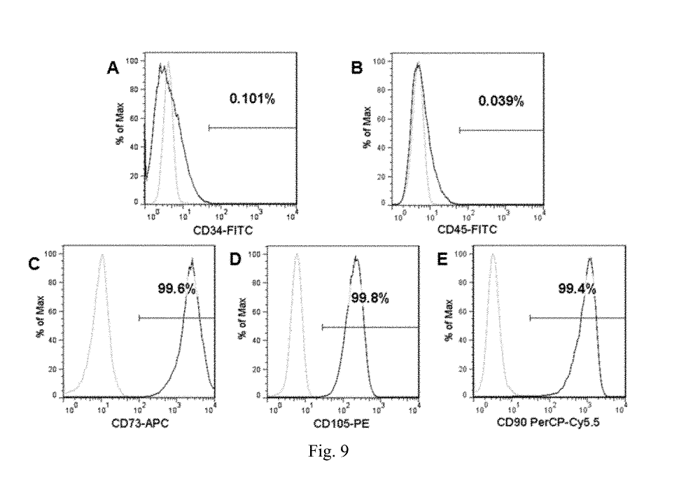

### DETAILED DESCRIPTION

### Example

A method for preparing a mesenchymal stem cell preparation by using an arthroscopic flushing solution of the present disclosure includes:

1) collecting a flushing solution in an arthroscopic surgery: the flushing solution was collected by a sterile collection bottle during the arthroscopic surgery and stored for use;
2) extracting and expanding stem cells: the collected arthroscopic flushing solution was dispensed (1000 ml/flask) on a clean bench and centrifuged at 1200 rpm for 15 min. A supernatant was discarded and DMEM medium containing 2% by weight of serum replacement was added to a resulting precipitate to resuspend the cells, which were then inoculated into a T25 culture flask. The medium was replaced after 48 h for the first time and was then replaced every 3 days for 12 days to 16 days to obtain adherent cells. When cell clones were in contact with each other, the cells were passaged. After passage, the cells were purified and expanded, and the 6th passage cells were used for subsequent stem cell identification and preparation preparing;
3) identifying characteristics of the stem cells: a proliferative ability of the stem cells was detected by using CCK-8 reagent; induction solutions were used for adipogenic, osteogenic, and chondrogenic induction of the stem cells, and a multi-directional differentiation ability of the stem cells was identified by staining; the stem cell surface markers (CD34⁻, CD45⁻, CD73⁺, CD90⁺, and CD105⁺) were analyzed by flow cytometry to identify the stem cell-specific antigens;
4) establishing processing standards for preparing stem cell preparations: standard operating process for preparing stem cell preparations including collection, isolation, purification, expansion, and passage of the stem cells, establishment of stem cell lines, directional differentiation into functional cells, selection criteria and use of culture media, excipients, and packaging materials, cell cryopreservation, resuscitation, dispensing, and labeling, and residue removal, as well as standard operating procedure (SOP) for each process were established.
5) preparing an arthroscopic flushing solution-derived stem cell preparation: the resulting stem cells were dispensed in a serum-free medium and storage additives thereof at a concentration of 1×10⁷ cells/ml and were stored in a cell bank which was divided into a main cell bank and a working cell bank, and autologous cell reinfusion could rule out the occurrence of immunogenic reactions;
6) controlling quality of the stem cell preparation: comprehensive tests of cell quality, safety, and efficacy were performed including tests of stem cell survival rate and growth activity, purity and homogeneity, sterility test and mycoplasma detection, intracellular exogenous pathogenic factor detection, endotoxin detection, and detection of residual amounts of culture medium and other additive components according to “Guidelines for Quality Control and Preclinical Research of Stem Cell Preparation (Trial)” issued by China on Aug. 21, 2015 with reference to guidelines for quality control of cell matrix and stem cell preparations at home and abroad, standards of release were established;
7) clinical assessment of the arthroscopic flushing solution-derived stem cell preparation: the prepared arthroscopic flushing solution-derived stem cell preparation was injected into the joint cavity of a patient by an autologous reinfusion in outpatient service for the regenerative repair of a damaged tissue.

1. A method for preparing a mesenchymal stem cell preparation by using an arthroscopic flushing solution, comprising the following steps:
1) collecting a flushing solution in an arthroscopic surgery: collecting the flushing solution by a sterile collection bottle during the arthroscopic surgery and storing the flushing solution for use;
2) extracting and expanding stem cells: dispensing the collected arthroscopic flushing solution on a clean bench and centrifuging the arthroscopic flushing solution, discarding a supernatant, adding DMEM medium containing 2% by weight of serum replacement to a resulting precipitate and resuspending the cells, inoculating the cells into a culture flask, replacing the medium for the first time after 45 h to 50 h, and then replacing the medium every 3 days for 12 days to 16 days to obtain adherent cells, passaging the cells when cell clones are in contact with each other; purifying and expanding the cells after passage, using the 6th passage cells for subsequent steps;
3) identifying characteristics of the stem cells: detecting a proliferative ability of the stem cells by using CCK-8 reagent; using induction solutions for adipogenic, osteogenic, and chondrogenic induction of the stem cells, identifying a multi-directional differentiation ability of the stem cells by staining; identifying mesenchymal stem cell-specific antigens by analyzing stem cell surface markers CD34⁻, CD45⁻, CD73⁺, CD90⁺, and CD105⁺ with the cells being negative when expressions of CD34⁻ and CD45⁻ are less than 1% and the cells being positive when expressions of CD73⁺, CD90⁺, and CD105⁺ are greater than 95%;
4) preparing a stem cell preparation: dispensing the resulting stem cells in a serum-free medium and storage additives thereof at a concentration of 1×10⁶ cells/ml to 1×10⁸ cells/ml and storing the stem cell preparation in a cell bank;
5) controlling quality of the stem cell preparation: performing tests selected from the group consisting of stem cell survival rate and growth activity, purity and homogeneity, sterility test and mycoplasma detection, detection of intracellular exogenous pathogenic factors, endotoxin detection, and detection of residual amounts of culture medium and other additive components according to “Guidelines for Quality Control and Preclinical Research of Stem Cell Preparation (Trial)” issued by China on Aug. 21, 2015 with all qualified preparations as finished products.
